(19) United States
(12) Patent Application Publication
(10) Pub. No.: US 2017/0035350 A1
(43) Pub. Date: Feb. 9, 2017

(54) SYSTEM AND METHOD FOR DETECTING BRUXISM

(71) Applicant: Michiel Allessie, Broek in Waterland (NL)

(72) Inventor: Michiel Allessie, Broek in Waterland (NL)

(73) Assignee: Bruxlab BV, Broek in Waterland (NL)

(21) Appl. No.: 14/817,252

(22) Filed: Aug. 4, 2015

(51) Int. Cl. A61B 5/00; A61B 5/11

(52) U.S. Cl. CPC A61B 5/4557; A61B 5/6814; A61B 5/0024; A61B 5/11; A61B 2562/0219

(57) ABSTRACT

The invention discloses a system and a method for the detection of sleep bruxism, the system comprising two sensor devices, the first sensor device is attached to the skin of the left masseter muscle of the patient and a second sensor device is attached to the skin of the right masseter muscle. The sensor devices each comprise an accelerometer, a processor module arranged for processing detection signals of the accelerometer and transmitting them to the processed signals to a wireless transceiver. The wireless transceiver is arranged for communicating the processed signals to a remote processor unit. An electronic circuitry comprises the accelerometer, the processor module and the wireless transceiver and is powered by a power source. The system further comprising a remote processor unit running software arranged for analyzing the communicated processed signals.

### TECHNICAL FIELD

The present invention relates to a systems and methods for detecting bruxism.

### BACKGROUND

The present invention relates to a system, a device and a method for the detection of bruxism. The term bruxism covers in this respect the abnormal excessive and non-functional nocturnal or subconscious grinding of teeth and jaw or tooth clenching (hereinafter referred to as clenching).

At a minimum, bruxism will typically result in excessive tooth wear and periodontal problems. Unfortunately in many cases this bruxing action not only damages the teeth themselves, but also the supporting structure of the teeth including both the hard bony material and the soft tissue. As a result, in more extreme cases these disorders lead to temporomandibular disorders, jaw displacement, stiff neck, and severe headaches.

Whenever the word patient is used hereinafter, it should be understood that this term implies any person, whether he or she is ill, suffering, in need for a treatment, hospitalized or is in none of these conditions. Whenever the word “his” or “he” is used, one may read also “her” or “she” respectively.

Bruxism is often classified as either awake bruxism or sleep bruxism. Awake bruxism is the condition wherein a patient habitually clenches his teeth and jaw when awake, usually without any teeth grinding. Most patients will do this subconsciously while concentrating. Sleep bruxism is the condition wherein a patient subconsciously grinds his teeth and squeezes his jaw muscles in his sleep. Partners may hear the grinding, which can be noisy.

A first step in treatment of bruxism is to establish if the patient is actually showing symptoms of bruxism and to assess the severity of bruxism. For this purpose it is important to know when bruxism occurs and to establish the frequency and level of the jaw motion related to bruxism. Especially when asleep a patient is not able to register consciously whether or not bruxism occurs, moreover bruxism occurs mostly unconsciously. Several attempts have been made to detect and measure bruxism by means of technical aids. Hereinafter some prior art solutions are discussed.

U.S. Patent application No. US 2012/0048013 A1 by National University Corporation Tokyo Medical and Dental University discloses a jaw motion measuring system that can attach a simple detector and can carry out measurement of jaw motion. The jaw motion measuring system includes: an acceleration detector having at least an acceleration sensor that senses accelerations of three-axis directions, a flexible wire whose one end is connected to the acceleration sensor, an output terminal that is connected to another end of the flexible wire and outputs acceleration data of the three-axis directions, and an attachment portion that attaches the acceleration sensor to a chin portion of a lower jaw; and a jaw motion measuring device that acquires acceleration data corresponding to jaw motion from the acceleration detector, and carries out correction, with respect to the acquired acceleration data, of errors due to the acceleration sensor, and measures jaw motion expressed by acceleration waveforms.

U.S. Pat. No. 4,934,378 discloses a system for monitoring bruxism by measuring the electrical signals emitted by the jaw muscles. The monitoring apparatus is mounted on a probe that is inserted into one of the user's ear channels. When the system detects jaw muscle activity associated with bruxism, it alerts the user, for example by emitting an audible tone or a prerecorded message.

U.S. Pat. No. 4,715,367 discloses a behavioral modification device which may be used to detect and treat snoring, bruxism, and sleep apnea. The patent discloses the use of pressure sensors mounted on either side of the forehead and actuated by flexing the temporal muscles. The patent also discloses using microphones to sense breathing and snoring. The output of the system is a regulatable electric shock applied to the user through a neck collar.

Although the above described systems have been devised to detect a patient's tendency towards bruxism, these systems have typically met only limited success for a variety of reasons. For example, many systems are unreasonably uncomfortable, making normal sleep or day time use impossible. Moreover, the current systems are not able to detect accurately if the bruxism occurs during a real sleep period of the patient or for example during a short interrupt (wakeup) of the sleep period.

### SUMMARY

It is an object of the invention to provide a solution for detection of bruxism which is accurate, non-obtrusive, reduces costs and is convenient to use by a user. The object is achieved by using at least two sensor devices each comprising an accelerometer. A first sensor device is attached to the skin of the left masseter muscle for the detection of acceleration of the left masseter muscle and a second sensor device is attached to the right masseter muscle for the detection of acceleration of the right masseter muscle. The sensor devices further comprise transceivers which communicate data related to the detected acceleration wirelessly to a processor unit. The communicated data are processed by the processor unit which is for example comprised in a smart phone or a computer. When the processed data of the left and the right masseter muscles indicate that the acceleration of the left and the right masseter muscles is substantially equal, a positive detection of bruxism is registered by the processor.

### DESCRIPTION OF DRAWINGS

The figures show views of embodiments in accordance with the present invention.

FIG. 1 shows a representation of the invented system.

FIG. 2a and FIG. 2b show side views of the patient's head, without skin shown, with the position of invented sensor displayed.

FIG. 3 shows a representation of a patient's head with the invented sensor device attached to the patient's skin.

FIG. 4 shows a schematic overview of the electronics of the sensor device according to the invention.

FIG. 5 shows a diagram representing the method according to the invention.

### DETAILED DESCRIPTION

As stated before, bruxism comprises tooth grinding and clenching. Tooth grinding is noticeable in two manners. The first one being that the teeth of the lower jaw move mostly sideways back and forth over the teeth of the upper jaw, without the purpose of actually grinding food. The second one being that the tooth grinding causes typical sounds associated with tooth grinding. Applicant has disclosed a method already for detecting and analyzing these sounds, and using the results to establish if the patient is suffering from (sleep) bruxism. The patent application with number U.S. Ser. No. 14/504,452 in relation to this referred disclosure by applicant is incorporated herewith.

Bruxism is measured by comparing the acquired measuring data with the so called Bruxism Episode Index (abbreviated as BEI). The current way of measuring the data is by measuring electromyography (abbreviated as EMG) of the muscles of the patient's jaws. The American Academy of Sleepmedicine (abbreviated as AASM) defines episodes of bruxism as follows:
PHASIC: at least three EMG bursts lasting between 0.25 seconds and 2 seconds
TONIC: One EMG burst lasting more than 2 seconds,
MIXED: a mix of phasic and tonic bursts.
   Only burst that follow within 3 seconds are considered to be part of the same episode.

The invention proposes to use acceleration of the masseter muscles as a way to determine the BEI instead of using EMG. Processed data of the left and right masseter may be used to determine if a bruxism episode has occurred. By using an invented and proprietary algorithms to translate the measured acceleration into information related to bruxism, a calculation of the BEI is possible. To increase accuracy and/or to verify this information, our method for determining bruxism by audio recording as described in U.S. Ser. No. 14/504,452 may be combined with the present invention.

Our proprietary algorithms are based on our discovery that a first phenomenon may serve very well as indication of clenching as part of bruxism. The first phenomenon being that the left and the right masseter muscle of a patient contract (almost) exactly on the same time (simultaneously) with a similar intensity. Our invention comprises that these contractions are measure by our invented sensor devices which each comprise an accelerometer. By attaching a first sensor device (hereinafter also referred to as a “cheek sensor”) to the left masseter muscle and a second cheek sensor to the right masseter muscle, acceleration in an outward direction on the x-axis (outward in relation to the patient's head) of the first cheek sensor may be compared to an outward acceleration in opposite direction of the second cheek sensor. If the comparison indicates that both measured accelerations (be it in opposite direction) are simultaneous or almost simultaneous, we can assume that bruxism occurs. By logging the measured accelerations, it is possible to add a BEI to the occurrence of bruxism.

We also discovered that a second phenomenon supports the indication of bruxism, which comprises that the simultaneous contraction of the left and the right masseter muscle is accompanied by or immediately preceded by closing of the patient's mouth.

A further improvement of our invention comprises that we may filter out (temporary) wake states of the patient during a sleeping period, or determine that the patient is asleep, which would support the indication of sleep bruxism. Interruption of sleep may for example give acceleration readings of the patient's body moving, instead of only the patient's jaws moving or masseter muscles contracting. The filter is based on any combination of the following conditions.

A first condition comprises that acceleration data are designated as not relating to (sleep) bruxism if the acceleration values are beyond a determined range. A too low acceleration on the y-axis as measured by one or both of the cheek sensors may indicate for example a slow head movement in upward direction with a relatively stable jaw position. A unidirectional lateral acceleration as measured by the cheek sensors and/or the chin sensor may indicate a movement of the patient heads to the side or a movement of the patient's whole body from one side to another. Identifying these movements of the patient's body or head may indicate a wake or a sleep status of patient. The prolonged absence of these movements may in particular indicate a patient's sleep status.

A second condition comprises that acceleration data are designated as not relating to (sleep) bruxism if substantially continuous duration of the acceleration as measured by any of the sensor devices beyond a determined range are combined with acceleration values within a determined range. On the other hand certain acceleration values together with a certain duration of the acceleration may indicate that the patient is asleep. For example accelerations with low values but lasting relatively long, may indicate a relatively large movement of the patient's body or head. On the other hand a relatively high acceleration value in combination with a very short duration may for example indicate a hypnic jerk (or sleep twitch) of the body or head. Twitches may for example be an indication of the patient falling asleep when followed by a general absence of measured acceleration, or an indication of awakening, for example when followed by measured accelerations.

Yet a further improvement of our invention comprises that our invented sensors devices, system and method may distinguish movements of the body or head from specific contractions of the patient's masseter muscles or the lower jaw. For example an acceleration value of 0.1 m/s2 in only a direction to the left on the x-axis because of a movement of the patient's head to the left side may be increased by a contraction of the left masseter muscle, which may be 0.4 m/s2 for example. This acceleration adds up to the head acceleration, so the left cheek sensor measures an acceleration of 0.5 m/s2 in this case. In the case of an occurrence of bruxism the right masseter muscle would contract substantially at the same time with a similar intensity, so in the given example the contraction of the right masseter muscle would lead to an acceleration value of 0.4 m/s2 in opposite direction on the x-axis. At the same time however, the chin sensor may measure an acceleration of the jaw to the left on the x-axis. This value would be 0.1 m/s2, assuming that the jaw moves together with the head. This assumption is supported if a general absence of acceleration on the y-axis occurs, indicative a of a stable closed mouth status.

The invention is now described by the following aspects and embodiments, with reference to the figures.

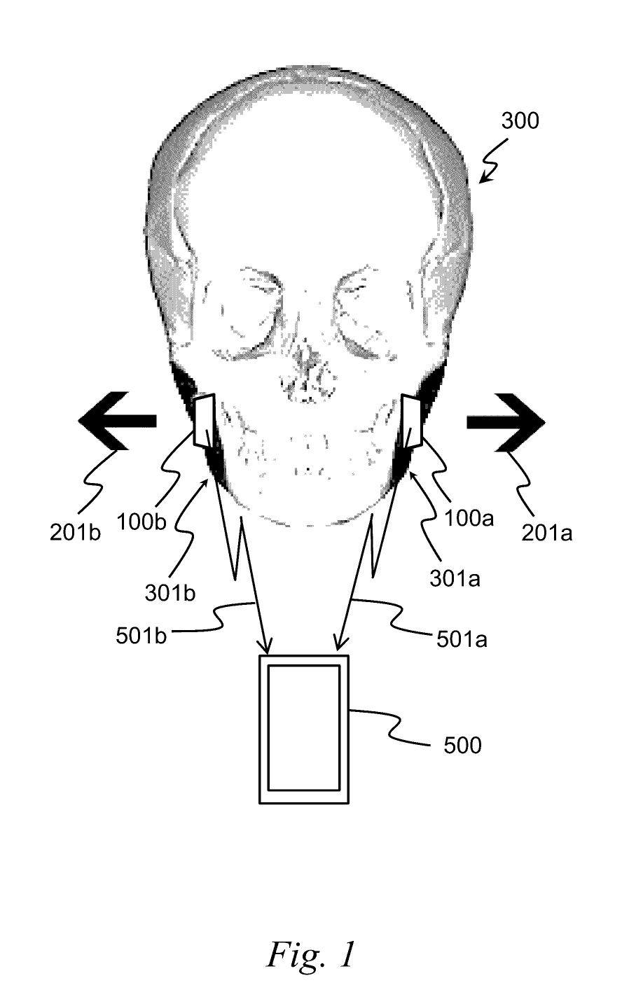

FIG. 1 shows a representation of the invented system. A front view of a patient's head 300 is shown without the skin, in order to show the position of the masseter muscle 301a,b. A first sensor device 100b is depicted as superimposed on the left masseter muscle 301a. In reality the sensor devices 100a,b will be attached to the skin covering the masseter muscles. On the right side of the patent's head 300 a second sensor device 100b is superimposed on the right masseter muscle 301b. When the patient clenches, the masseter muscles 301a,b contract and expand in outward direction (in other words they are getting thicker) as indicated by arrows 201a and 201b respectively. The arrows 201a and 201b therefore also indicate the acceleration direction, which is detected by sensor devices 100a and 100b respectively. The sideway directions, as indicated by arrows 201a,b, are directions on the x-axis and are opposite to each other. The opposite acceleration occurring substantially simultaneously and at about the same intensity are indicative of a bruxism episode. Arrows 501a and 501b represent a wireless connection from sensor devices 100a,b to an external device 500 comprising a processor for processing the wirelessly received signals of the sensor devices 100a,b.

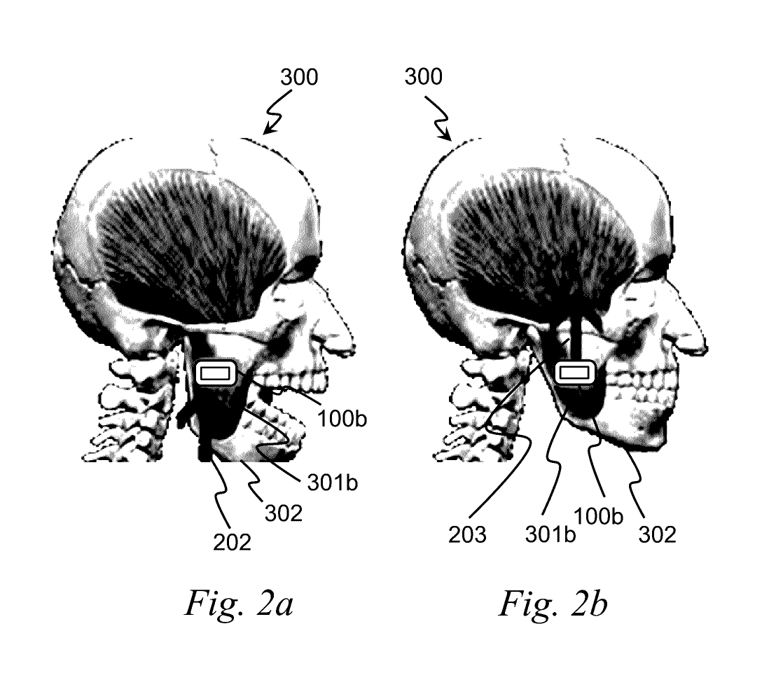

FIG. 2a and FIG. 2b show side views of the patient's head 300. The head 300 is shown without the skin in order to show the position of the right masseter muscle 301b. The second sensor device 100b is depicted as superimposed on the right masseter muscle 301b. Also here, in reality the sensor devices 100b will be attached to the skin covering the masseter muscles. FIG. 2a shows the head 300 with open mouth i.e. a lowered lower jaw 302. When the lower jaw 302 is moved upwards i.e. upwards on the y-axis, as indicated by arrow 202, by contraction of masseter muscles 301a,b, an upward directional movement of a part of the masseter muscles 301a,b occurs as well. The acceleration of muscle tissue of masseter muscles 301a,b in upward direction is detected by sensor devices 100a,b. When, as depicted in FIG. 2b, the patient's mouth is already closed, the masseter muscles 301a,b may still be contracted. This is typical for clenching and indication for a bruxism episode. The contraction of the masseter muscles 301a,b also results in an upward acceleration, as indicated by arrow 203, of the tissue of masseter muscles 301a,b and therefore will be detected by the sensor devices 100a,b. Typically when the contraction of the masseter muscles 301a,b is taking place with mouth closed (i.e. clenching) and is preceded by a closing of the mouth (i.e. the lower jaw 302 moving upward), this is an indication of a bruxism episode.

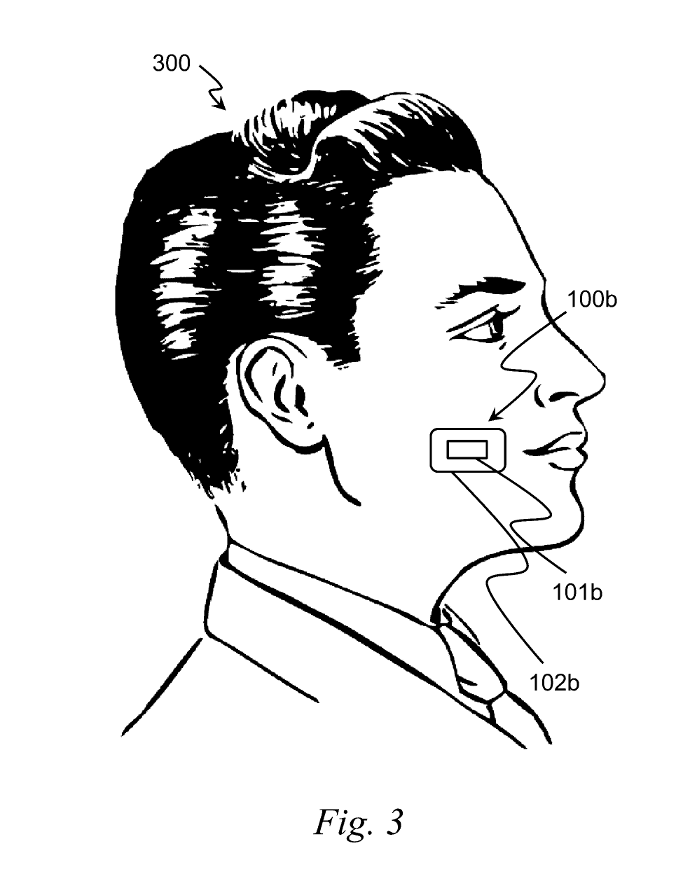

FIG. 3 shows a representation of a patient's head 300. A sensor device 100b according to the invention, is attached to the patient's skin on top of the masseter muscle, preferably at a position where the masseter muscle is best felt through the skin, when contracting. Attaching the sensor device 100b is done by sticking flexible part 102b to the skin using a (body) adhesive, preferably a skin friendly adhesive, i.e. compliant with ISO 10993, which is easily applicable and easily removable and which prevents the sensor device to release unwantedly. The electronics, such as the accelerometer, wireless communication module and battery is housed in housing 101b which may be integrated in the flexible part 102b.

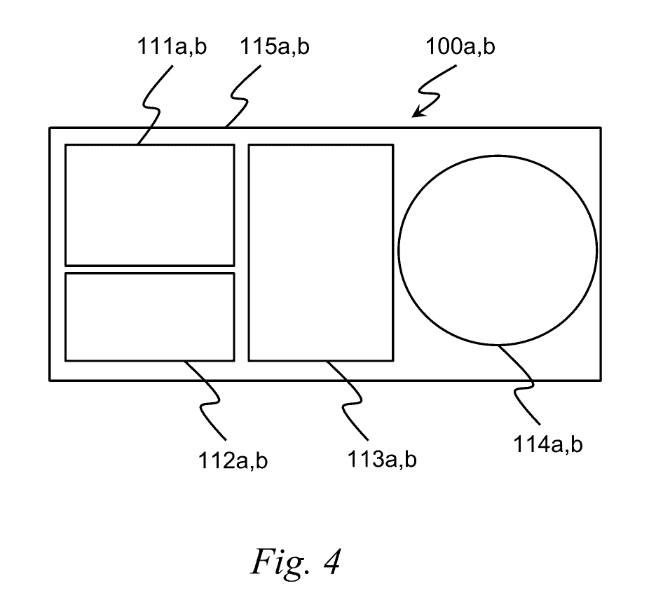

FIG. 4 shows a schematic overview of the electronics of the sensor device 100a,b according to the invention. Accelerometer 113a is arranged for detecting and measuring acceleration of the sensor device 100a and for sending the detected signals to processor module 112a. Processor module 112a is arranged for processing the signals received form the accelerometer 113a, and for transmitting the processed signals to transceiver 111a. Transceiver 111a is arranged for transmitting the processed signals wirelessly to an external device which is arranged for receiving the wirelessly transmitted processed signals and to process these signals. Battery 114a is provided for powering the sensor device. The above mentioned components are mounted on a printed circuit board 115a. A similar setup (not shown) is provided for sensor device 100b, wherein accelerometer 113b, processor module 112b, transceiver 111b, battery 114b and printed circuit board 115b are referenced as the relevant components. No wires are shown in the diagram, but a skilled person will be able to connect the components in a functional manner.

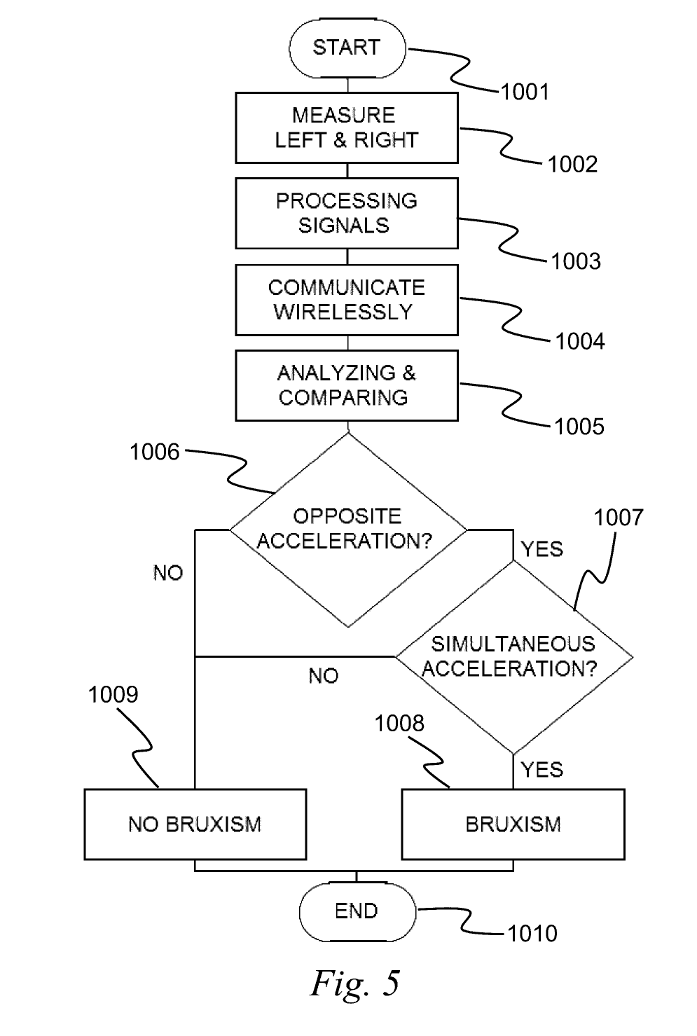

FIG. 5 shows a diagram representing the method according to the invention. The method steps are described as follows:

1001: The method starts;

1002: a first sensor device 100a comprising a first accelerometer 113a which measures acceleration of the left masseter muscle 301a of the patient, and translates the acceleration into first acceleration signals. A second sensor device 100b comprising a second accelerometer 113b which measures acceleration of the right masseter muscle 301b of the patient and translates the acceleration into second acceleration signals; 1003: a first processor module 112a of the sensor device 100a processes the detected first acceleration signals. A second processor module 112b of the sensor device 100b processes the detected second acceleration signals;

1004: a first wireless transceiver 111a communicates the processed first acceleration signals to a remote device 500 comprising a processor unit running analyzing software. A second wireless transceiver 111b communicates the processed second acceleration signals to the remote device 500;

1005: the processor unit of the remote device 500 analyzes the processed first and second acceleration signals and compares the analyzed first acceleration signals with the analyzed second acceleration signals;

1006: The processor unit of the remote device 500 determines occurrence of bruxism episodes when the analyzed first acceleration signals indicate an acceleration of the left masseter muscle 301a in an opposite direction of the acceleration of the right masseter muscle 301b as indicated by the analyzed second acceleration signals;

1007: If opposite acceleration is determined, then the processor unit of the remote device 500 determines whether or not the indicated acceleration of the left masseter muscle 301a occurs substantially simultaneously with the indicated acceleration of the right masseter muscle;

1008: If simultaneous acceleration occurs, then a bruxism episode is present;

1009: If no opposite acceleration and no simultaneous acceleration is determined, then a bruxism episode is not present;

1010: The method ends, or starts again at 1001 in a continuous loop.

In a first aspect of the present invention a system for the detection of bruxism is provided, the system comprising at least two sensor devices arranged for being attached to the skin of a patient, a first sensor devices of the at least two sensor devices arranged for being attached to the skin covering a left masseter muscle of the patient and a second sensor device of the at least two sensor devices arranged for being attached to the skin covering a right masseter muscle of the patient, wherein the at least two sensor devices each comprising:
an accelerometer arranged for measuring acceleration of the sensor device;
a processor module arranged for processing detection signals of the accelerometer;
the processor module arranged for transmitting the processed signals to a wireless transceiver;
the wireless transceiver arranged for communicating the processed signals to a remote processor unit;
an electronic circuitry comprising the accelerometer, the processor module and the wireless transceiver;
a power source arranged for providing power to the electronic circuitry, the system further comprising a remote processor unit running software arranged for analyzing the communicated processed signals.

Further embodiments of the first aspect comprise the following.

A sensor device for the detection of bruxism for use in the system, wherein the sensor device may comprise:
a housing arranged for housing an electronic circuit, the housing further comprising a sticker part arranged for temporary attaching the sensor device to the skin of the patient substantially in the area of the masseter muscle;
an accelerometer arranged for measuring acceleration of the sensor device;
a processor module arranged for processing detection signals of the accelerometer;
the processor module arranged for transmitting the processed signals to a wireless transceiver;
the wireless transceiver arranged for communicating the processed signals to a remote processor unit;
an electronic circuitry comprising the accelerometer, the processor module and the wireless transceiver;
a power source arranged for providing power to the electronic circuitry.

A sensor device, wherein the accelerometer may be a multi-axis accelerometer.

In a second aspect of the invention a method for detecting bruxism of a patient is provided, said method comprising the steps of:
a first sensor device comprising a first accelerometer measuring acceleration of the left masseter muscle of the patient and translating the acceleration into first acceleration signals;
a second sensor device comprising a second accelerometer measuring acceleration of the right masseter muscle of the patient and translating the acceleration into second acceleration signals;
a first processor module of the first sensor device processing the first acceleration signals of the first accelerometer;
a second processor module of the second sensor device processing the second acceleration signals of the second accelerometer;
a first wireless transceiver of the first sensor device communicating the processed first acceleration signals to a remote device comprising a processor unit running analyzing software;
a second wireless transceiver of second sensor device communicating the processed second acceleration signals to the remote device;
the processor unit of the remote device analyzing the processed first and second acceleration signals and comparing the analyzed first acceleration signals with the analyzed second acceleration signals;

The processor unit of the remote device determining occurrence of bruxism episodes when the analyzed first acceleration signals indicate an acceleration of the left masseter muscle in an opposite direction of the acceleration of the right masseter muscle as indicated by the analyzed second acceleration signals, and the indicated acceleration of the left masseter muscle occurs substantially simultaneously with the indicated acceleration of the right masseter muscle.

Further embodiments of the second aspect comprise the following.

The method may comprise that a further confirmation of the occurrence of bruxism comprises a detection by the first and/or second sensor device of an upward acceleration of the left and/or right masseter muscle respectively, immediately preceding the simultaneous opposite direction of the acceleration of the left and the right masseter muscles.

Bruxism may comprise sleep bruxism.

The term “substantially” herein, such as in “substantially . . . ” etc., will be understood by the person skilled in the art. In embodiments the adjective substantially may be removed. Where applicable, the term “substantially” may also include embodiments with “entirely”, “completely”, “all”, etc. Where applicable, the term “substantially” may also relate to 90% or higher, such as 95% or higher, especially 99% or higher, including 100%. The term “comprise” includes also embodiments wherein the term “comprises” means “consists of.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “to comprise” and its conjugations does not exclude the presence of elements or steps other than those stated in a claim. The term “and/or” includes any and all combinations of one or more of the associated listed items. The article “a” or “an” preceding an element does not exclude the presence of a plurality of such elements. The article “the” preceding an element does not exclude the presence of a plurality of such elements. In the device claim enumerating several means, several of these means may be embodied by one and the same item of hardware. The mere fact that certain measures are recited in mutually different dependent claims does not indicate that a combination of these measures cannot be used to advantage.

1. A system for the detection of bruxism, the system comprising at least two sensor devices arranged for being attached to the skin of a patient, a first sensor devices of the at least two sensor devices arranged for being attached to the skin covering a left masseter muscle of the patient and a second sensor device of the at least two sensor devices arranged for being attached to the skin covering a right masseter muscle of the patient, wherein the at least two sensor devices each comprising:
an accelerometer arranged for measuring acceleration of the sensor device;
a processor module arranged for processing detection signals of the accelerometer;
the processor module arranged for transmitting the processed signals to a wireless transceiver;
the wireless transceiver arranged for communicating the processed signals to a remote processor unit;
an electronic circuitry comprising the accelerometer, the processor module and the wireless transceiver;
a power source arranged for providing power to the electronic circuitry, the system further comprising a remote processor unit running software arranged for analyzing the communicated processed signals.

2. A sensor device for the detection of bruxism for use in a system as claimed in claim 1, wherein the sensor device comprises:
a housing arranged for housing an electronic circuit, the housing further comprising a sticker part arranged for temporary attaching the sensor device to the skin of the patient substantially in the area of the masseter muscle;
an accelerometer arranged for measuring acceleration of the sensor device;
a processor module arranged for processing detection signals of the accelerometer;
the processor module arranged for transmitting the processed signals to a wireless transceiver;
the wireless transceiver arranged for communicating the processed signals to a remote processor unit;
an electronic circuitry comprising the accelerometer, the processor module and the wireless transceiver;
a power source arranged for providing power to the electronic circuitry.

3. A sensor device according to claim 2, wherein the accelerometer is a multi-axis accelerometer.

4. A method for detecting bruxism of a patient, said method comprising the steps of:
A. a first sensor device comprising a first accelerometer measuring acceleration of the left masseter muscle of the patient and translating the acceleration into first acceleration signals;
B. a second sensor device comprising a second accelerometer measuring acceleration of the right masseter muscle of the patient and translating the acceleration into second acceleration signals;
C. a first processor module of the first sensor device processing the first acceleration signals of the first accelerometer;
D. a second processor module of the second sensor device processing the second acceleration signals of the second accelerometer;
E. a first wireless transceiver of the first sensor device communicating the processed first acceleration signals to a remote device comprising a processor unit running analyzing software;
F. a second wireless transceiver of second sensor device communicating the processed second acceleration signals to the remote device;
G. the processor unit of the remote device analyzing the processed first and second acceleration signals and comparing the analyzed first acceleration signals with the analyzed second acceleration signals;
H. The processor unit of the remote device determining occurrence of bruxism episodes when the analyzed first acceleration signals indicate an acceleration of the left masseter muscle in an opposite direction of the acceleration of the right masseter muscle as indicated by the analyzed second acceleration signals, and the indicated acceleration of the left masseter muscle occurs substantially simultaneously with the indicated acceleration of the right masseter muscle.

5. A method according to claim 4, wherein a further confirmation of the occurrence of bruxism comprises a detection by the first and/or second sensor device of an upward acceleration of the left and/or right masseter muscle respectively, immediately preceding the simultaneous opposite direction of the acceleration of the left and the right masseter muscles.

6. A method according to claim 4, wherein bruxism comprises sleep bruxism.
